(19) United States
(12) Patent Application Publication
(10) Pub. No.: US 2026/0345027 A1
(43) Pub. Date: Nov. 5, 2026

(54) IMPLANTABLE PULMONARY FUNCTION MONITOR

(71) Applicant: Medtronic, Inc., Minneapolis, MN (US)

(72) Inventors: Trent M. Fischer, St. Paul, MN (US); Paul G. Krause, Mahtomedi, MN (US); Nicholas H. Finstrom, Coon Rapids, MN (US); Catherine R. Condie, Shoreview, MN (US)

(21) Appl. No.: 19/661,763

(22) Filed: Apr. 29, 2026

Related U.S. Application Data
(60) Provisional application No. 63/798,270, filed on May 1, 2025.

(51) Int. Cl. A61B 5/08; A61B 5/00

(52) U.S. Cl. CPC A61B 5/08; A61B 5/0031; A61B 5/4884; A61B 5/686; A61B 5/7221

(57) ABSTRACT

A system includes an implantable medical device (IMD) comprising sensing circuitry configured to sense one or more physiological signals of a patient; and processing circuitry configured to: prompt, via a user interface, the patient to perform an exercise according to a periodic schedule; control the sensing circuitry to sense the one or more physiological signals of the patient while the patient performs the exercise; based on the one or more physiological signals sensed while the patient performs the exercise, determine a pulmonary function of the patient; and output an indication indicative of the pulmonary function of the patient for user review.

### RELATED APPLICATIONS

This application claims the benefit of U.S. Provisional Application Serial Number 63/798,270, filed May 1, 2025, the entire contents of which are incorporated herein by reference.

### FIELD

This disclosure generally relates to medical devices and, more particularly, to monitoring device systems.

### BACKGROUND

A variety of implantable medical devices (IMD) have been clinically implanted or proposed for therapeutically treating or monitoring cardiac, lung, neurological, and/or other conditions of a patient. IMDs may monitor physiological signals of the patient. For example, an IMD may monitor a condition of the patient. Patient conditions can change over time as the condition worsens or as therapy and lifestyle adjustments alleviate symptoms. In general, using physiological signals, such devices facilitate monitoring and evaluating patient health over a number of months or years as the patient condition changes, outside of a clinic setting.

### SUMMARY

In general, this disclosure describes techniques for monitoring pulmonary function of patients, e.g., patients with chronic obstructive pulmonary disease (COPD). More specifically, this disclosure describes techniques for sensing one or more physiological signals and determining pulmonary function of a patient. This disclosure additionally describes techniques for outputting pulmonary function information for user review.

A system may include an implantable medical device (IMD) configured to sense one or more physiological signals of a patient. In some examples, the system may prompt the patient to perform an exercise, e.g., a breathing exercise, according to a periodic schedule. While the patient performs the exercise, the IMD senses the one or more physiological signals. Based on the one or more physiological signals, the system determines a pulmonary function of the patient. The system outputs an indication indicative of the pulmonary function of the patient. In some examples, the pulmonary function information may include, for example, trends in pulmonary function, one or more indices of pulmonary function, e.g., a forced expiratory volume in one second (FEV1), a forced vital capacity (FVC), an FEV1 to FVC ratio, a forced mid-expiratory flow (FEF), and/or a peak expiratory flow (PEF), suggestions to adjust treatment, e.g., an indication to adjust a medication dosage, or an indication to take a rescue medication, e.g., use a rescue inhaler.

The techniques, systems, and methods of this disclosure may advantageously allow a user to monitor a patient condition over time. Current methods for diagnosing and initially assessing COPD include spirometry testing, which many patients find inconvenient and uncomfortable. Most patients perform a spirometry test only once during a clinician visit upon being diagnosed with COPD. Patients do not generally perform spirometry testing at home to monitor progression of COPD. To monitor COPD progression, current methods include monitoring changes in symptoms. However, monitoring for changes in symptoms is less accurate and more subjective than spirometry testing. Therefore, the pulmonary function of the patient may worsen or improve without user knowledge. By sensing one or more physiological signals of the patient during a patient exercise, such as a deep breathing exercise, according to a periodic schedule, the techniques of this disclosure my facilitate longitudinal monitoring of patients with COPD. In some examples, by monitoring COPD according to the periodic schedule, the techniques of this disclosure may facilitate timely interventions, such as changes in patient medication or other forms of COPD management. By intervening in a relatively timely fashion, the techniques of this disclosure may improve patient comfort and outcomes.

In some examples, the techniques of this disclosure may advantageously allow a user to assess efficacy associated with changes in medication, e.g., bronchodilation medication. As an example, the techniques of this disclosure may include determining trends in pulmonary function, e.g., trends in one or more indices of pulmonary function. The systems of this disclosure may be configured to determine changes in pulmonary function associated with changes in medication. By assessing efficacy associated with changes in medication, the techniques of this disclosure may facilitate timely adjustments in medication and may allow the clinician to monitor changes in the COPD condition and/or overall health of the patient.

Additionally, in some examples, patients may find the exercises described herein less physically taxing than traditional spirometry testing. Patients may be more likely to perform the exercises described herein than to perform spirometry testing, thereby improving patient compliance and facilitating pulmonary function data acquisition.

Spirometry testing often facilitates lung-clearing. However, as patients do not generally perform regular spirometry testing outside of a clinic or hospital setting, patients do not currently perform lung-clearing exercises as frequently as may be desired and/or efficacious. In some examples, the exercises described herein, e.g., breathing exercises, may aid the patient in clearing their lungs, which may improve patient comfort and outcomes.

In some examples, in addition or alternatively to determining pulmonary function based on one or more physiological signals sensed while the patient performs exercise(s), the techniques may include continuously, e.g., periodically, such as hourly, sensing the one or more physiological signals to determine pulmonary function. In some examples, the system may determine pulmonary function in response to determining one or more physiological signals meet one or more criteria. For example, if one or more physiological signals of the patient are indicative of a potential change in pulmonary function, the system may determine the pulmonary function of the patient. In some examples, the system may determine the pulmonary function of the patient based on a determination that an environmental condition meets one or more criteria. By determining the pulmonary function of the patient in response to one or more criteria being met, the techniques of this disclosure may result in determining pulmonary function when there are changes the pulmonary function of the patient that may benefit from changes in treatment. For example, if the patient is in an environment with relatively heavy particulate air pollution, e.g., a smoggy environment, resulting in worsened pulmonary function, the system may determine to output an indication to adjust a medication of the patient.

In some examples, the system may be configured to determine a quality associated with the one or more physiological signals before determining and/or outputting the indication of the pulmonary function. For example, the system may determine whether an effort of the patient associated with the one or more physiological signals meets a quality criterion. As another example, the system may determine whether the patient was in an appropriate posture during an exercise before determining the pulmonary function based on the one or more physiological signals. In some examples, by determining the one or more physiological signals meet one or more quality criterion, the techniques of this disclosure may result in more accurate determinations of pulmonary function than systems in which quality is not determined.

In one example, a system comprises: an implantable medical device (IMD) comprising sensing circuitry configured to sense one or more physiological signals of a patient; and processing circuitry configured to: prompt, via a user interface, the patient to perform an exercise according to a periodic schedule; control the sensing circuitry to sense the one or more physiological signals of the patient while the patient performs the exercise; based on the one or more physiological signals sensed while the patient performs the exercise, determine a pulmonary function of the patient; and output an indication indicative of the pulmonary function of the patient for user review.

In another example, a method comprises: prompting, by processing circuitry of a medical device system comprising an implantable medical device (IMD) and via a user interface, a patient to perform an exercise according to a periodic schedule; controlling, by the processing circuitry, sensing circuitry of the IMD to sense one or more physiological signals of the patient while the patient performs the exercise; based on the one or more physiological signals sensed while the patient performs the exercise, determining, by the processing circuitry, a pulmonary function of the patient; and outputting, by the processing circuitry, an indication indicative of the pulmonary function of the patient for user review.

In another example, a non-transitory computer-readable medium stores instructions that when executed cause processing circuitry to: prompting, via a user interface, a patient to perform an exercise according to a periodic schedule; control sensing circuitry of an implantable medical device (IMD) of a medical device system comprising the processing circuitry to sense one or more physiological signals of the patient while the patient performs the exercise; based on the one or more physiological signals sensed while the patient performs the exercise, determine a pulmonary function of the patient; and output an indication indicative of the pulmonary function of the patient for user review.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 illustrates the environment of an example medical device system in conjunction with a patient, in accordance with one or more techniques of this disclosure.

FIGS. 2A and 2B are conceptual diagrams illustrating example implantable medical devices that operate in accordance with one or more techniques of this disclosure.

FIG. 3 is a block diagram illustrating an example configuration of an implantable medical device that operates in accordance with one or more techniques of this disclosure.

FIG. 4 is a block diagram illustrating an example configuration of an external device that operates in accordance with one or more techniques of this disclosure.

FIG. 5 is a flow diagram illustrating an example operation for determining a pulmonary function of a patient based on one or more sensed physiological signals meeting one or more corresponding criteria, in accordance with one or more techniques of this disclosure.

FIG. 6 is a flow diagram illustrating an example operation for determining a pulmonary function of a patient based on signals sensed while the patient performed an exercise, in accordance with one or more techniques of this disclosure.

FIG. 7 is a flow diagram illustrating an example operation for determining a pulmonary function of a patient based on signals sensed according to a periodic schedule, in accordance with one or more techniques of this disclosure.

FIG. 8 is a flow diagram illustrating an example operation for determining an overall pulmonary function of a patient based on signals sensed while the patient performed a second exercise, in accordance with one or more techniques of this disclosure.

FIG. 9 is a flow diagram illustrating an example operation for determining whether to output an indication indicative of the pulmonary function to the patient, a clinician, a caregiver, or a combination thereof, in accordance with one or more techniques of this disclosure.

FIG. 10 is a flow diagram illustrating an example operation for determining whether to determine pulmonary function based on a quality level of the one or more physiological signals, in accordance with one or more techniques of this disclosure.

Like reference characters refer to like elements throughout the figures and description.

### DETAILED DESCRIPTION

A variety of types of implantable and external devices are configured to monitor health based on sensed electrocardiograms (ECGs) and, in some cases, other physiological signals. External devices that may be used to non-invasively sense and monitor ECGs and other physiological signals include wearable devices with electrodes configured to contact the skin of the patient, such as patches, watches, rings, necklaces, hearing aids, a wearable cardiac monitor or automated external defibrillator (AED), clothing, car seats, or bed linens. Such external devices may facilitate relatively longer-term monitoring of patient health during normal daily activities.

Implantable medical device (IMD) systems also sense and monitor physiological signals to monitor patient conditions, e.g., chronic obstructive pulmonary disease (COPD). Some IMDs do not provide therapy, such as implantable patient monitors. One example of such an IMD is the Reveal LINQ™ or LINQ II™ insertable cardiac monitors (ICMs), available from Medtronic, Inc., which may be inserted subcutaneously. Such IMDs may facilitate relatively longer-term continuous monitoring of patients during normal daily activities, and may periodically or on demand transmit collected data, e.g., data indicative of pulmonary function, to a remote monitoring system, such as the Medtronic CareLink™ Network via a home monitoring system or a smart phone application.

In some examples, a system including an IMD may be configured to sense one or more physiological signals of a patient. In some examples, the system may be configured to, via a user interface, prompt the patient to perform an exercise according to a periodic schedule, such as a breathing exercise, e.g., a seated deep breathing exercise, or a walking exercise, e.g., a 6 minute hallway walking test. The IMD may be configured to sense the one or more physiological signals while the patient performs the exercise. Based on the one or more physiological signals, the system may determine one or more indices of pulmonary function, e.g., a forced expiratory volume in one second (FEV1), a forced vital capacity (FVC), an FEV1 to FVC ratio, a forced mid-expiratory flow (FEF), a peak expiratory flow (PEF), and/or an overall health of the patient. In some examples, the breathing exercise may be indicative of pulmonary function, and the walking exercise may be indicative of an overall patient health.

The techniques, systems, and methods of this disclosure may advantageously facilitate longitudinal monitoring of COPD. Current methods for monitoring COPD include spirometry testing. Most patients perform a spirometry test during a clinician visit upon being diagnosed with COPD, but patients do not generally perform spirometry testing at home to monitor progression of COPD due to discomfort associated with spirometry testing. By sensing one or more physiological signals of the patient during a patient exercise, such as a deep breathing exercise, according to a periodic schedule, the techniques of this disclosure my facilitate longitudinal monitoring of patients with COPD. In some examples, by monitoring COPD according to the periodic schedule, the techniques of this disclosure may facilitate timely interventions, such as changes in patient medication or other forms of COPD management, thereby improving patient comfort and outcomes.

In some examples, in addition or alternatively to determining pulmonary function based on one or more physiological signals sensed while the patient performs exercise(s), the system may continuously, e.g., periodically, sense the one or more physiological signals to determine pulmonary function.

In some examples, the system may determine pulmonary function in response to determining whether one or more physiological signals meet one or more criteria. For example, if one or more physiological signals of the patient are indicative of a potential change in pulmonary function, the system may determine the pulmonary function of the patient. In some examples, the system may determine the pulmonary function of the patient based on a determination that an environmental condition meets one or more criteria. By determining the pulmonary function of the patient in response to one or more criteria being met, the techniques of this disclosure may result in determining pulmonary function when there are changes the pulmonary function of the patient that may benefit changes in treatment.

In some examples, the system may be configured to determine a quality associated with the one or more physiological signals before determining and/or outputting the indication of the pulmonary function. For example, the system may determine whether an effort of the patient associated with the one or more physiological signals meets a quality criterion. As another example, the system may determine whether the patient was in an appropriate posture during an exercise before determining the pulmonary function based on the one or more physiological signals. In some examples, by determining the one or more physiological signals meet one or more quality criterion, the techniques of this disclosure may result in accurate determinations of pulmonary function.

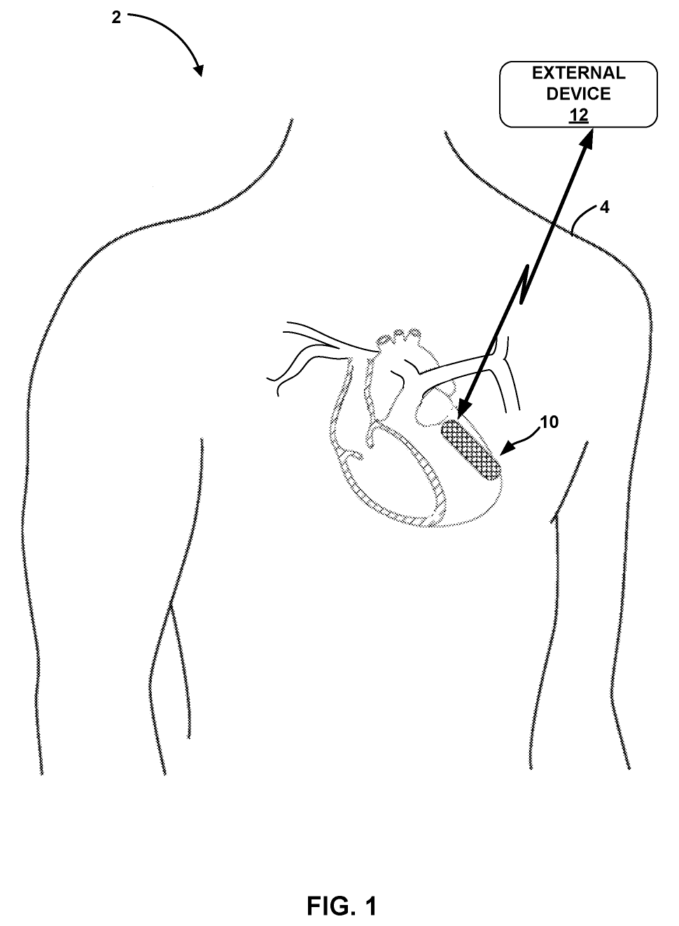

FIG. 1 illustrates the environment of an example medical device system 2 in conjunction with a patient 4, in accordance with one or more techniques of this disclosure. The example techniques may be used with one or more patient sensing devices, e.g., including an IMD 10, which may be in wireless communication with one or more computing devices, e.g., external device 12. In some examples, external device 12 may be in communication with a computing system via a network of system 2 (not depicted). Although not illustrated in FIG. 1, IMD 10 includes electrodes and/or other sensors to sense one or more physiological signals of patient 4 and may collect and store physiological signal data based on the one or more sensed physiological signals, e.g., an impedance measurement, an electromyography (EMG) signal, an electrocardiogram (ECG) signal, a heart rate signal, a photoplethysmography (PPG) signal, a tissue oxygen saturation signal, a peripheral oxygen saturation signal, an accelerometer signal, or a pressure signal.

Based on the one or more sensed physiological signals, system 2, e.g., IMD 10 and/or external device 12, may determine a pulmonary function of patient 4, e.g., one or more indices of pulmonary function. In some examples, system 2 determines the one or more indices of pulmonary function based the one or more sensed physiological signals using a calibration. In some examples, system 2 may be configured to calibrate the one or more sensed physiological signals with spirometry test measurements to determine the one or more indices of pulmonary function. In some examples, the calibration may be patient specific. In some examples, system 2 may calibrate the one or more sensed physiological signals with spirometry test measurements one time, e.g., during initial implantation and configuration of IMD 10, several times, e.g., every few weeks or months or yearly. In some examples, system 2 may not calibrate the one or more sensed physiological signals with spirometry test measurements. In some examples, IMD 10 senses the one or more physiological signals when patient 4 is performing an exercise, e.g., a breathing or walking exercise. In some examples, external device 12 prompts patient 4 to perform the exercise according to a periodic schedule. In some examples, one or more elements of system 2 may assess a quality associated with the one or more physiological signals. For example, system 2 may determine one or more of whether patient 4 was in a seated position during an exercise based on accelerometer data or a patient effort associated with the exercise based on one or more of the physiological signals, patient input, and/or one or more previous signals. If patient 4 was not in a seated position during the exercise or if the patient effort falls below a threshold, system 2 may determine the quality associated with the one or more physiological signals does not meet a quality criterion. System 2 may determine not to determine the pulmonary function and may prompt patient 4 to redo the exercise at a later time.

In some examples, system 2 additionally or alternatively determines the pulmonary function of patient 4 based on one or more physiological signals sensed continuously, e.g., hourly, and not corresponding to when patient 4 performs the exercise. In other words, system 2 may determine pulmonary function “in the background.”

In some examples, IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette, and be configured to sense one or more physiological signals from that position. In some examples, IMD 10 takes the form of the Reveal LINQ™ or LINQ II™ ICM.

Although described primarily in the context of examples in which IMD 10 takes the form of an ICM, the techniques of this disclosure may be implemented in systems including any one or more implantable or external medical devices, including monitors, pacemakers, defibrillators (e.g., subcutaneous or substernal), wearable external defibrillators (WAEDs), neurostimulators, drug pumps, patch monitors, or wearable physiological monitors, e.g., wrist, finger, or head wearable devices, such as smart watches, polar chest bands, or microphones. Examples with multiple IMDs or other sensing devices may be able to collect different data useable by system 2 to determine a pulmonary function and/or an overall health status of patient 4.

External device 12 may be a computing device with a display viewable by the user and an interface for providing input to external device 12 (i.e., a user input mechanism). External device 12 is configured for wireless communication with IMD 10. External device 12 retrieves sensed physiological data from IMD 10 that was collected and, in some examples, stored, by the IMD. In some examples, external device 12 takes the form of a personal computing device of patient 4. For example, external device 12 may take the form of a smartphone of patient 4. In some examples, external device 12 may be any computing device configured for wireless communication with IMD 10, such as a desktop, laptop, or tablet computer. External device 12 may communicate with IMD 10 via near-field communication technologies e.g., inductive coupling, NFC or other communication technologies operable at ranges less than 10–20 cm, and far-field communication technologies, e.g., radiofrequency telemetry according to the Bluetooth® or Bluetooth® Low Energy (BLE) protocols, or other communication technologies operable at ranges greater than near-field communication technologies. When external device 12 is configured for use by the clinician, external device 12 may be used to transmit instructions to IMD 10. The clinician may also configure and store operational parameters for IMD 10 with the aid of external device 12. In some examples, external device 12 assists the clinician in the configuration of IMD 10 by providing a system for identifying potentially beneficial operational parameter values.

External device 12 may be used to retrieve data from IMD 10. The retrieved data may include physiological signal data measured by IMD 10 based on one or more physiological signals sensed by IMD 10. For example, external device 12 may retrieve physiological signal data according to a periodic schedule, e.g., upon IMD 10 sensing the one or more physiological signals while patient 4 performs an exercise. In some examples, instead of or in addition to physiological signal data, external device may retrieve data indicating values of features determined by IMD 10 based on the one or more physiological signals.

Processing circuitry of system 2, e.g., of IMD 10, external device 12, and/or one or more other computing devices (not illustrated in FIG. 1) may be configured to perform the example techniques described herein for determine pulmonary function of patient 4 based on one or more physiological signals sensed by IMD 10. In some examples, one or more of the sensors, e.g., of IMD 10, may be implanted within patient 4, that is, implanted at least subcutaneously. In some examples, one or more of the sensors of IMD 10 may be located externally to patient 4, for example as part of a cuff or as a wearable device, e.g., a smart watch.

In some examples, IMD 10 transmits data to an external device 12 e.g., a smartphone of patient 4, which may may in some examples transmit the data to a computing system via a network. System 2 may be configured to process the data and notify the clinician and/or patient 4 of the pulmonary function of patient 4. In some examples, computing system 230 may additionally be configured to alert the clinician and/or patient 4 when an overall health of patient 4 has changed, e.g., when patient 4’s COPD has worsened or improved.

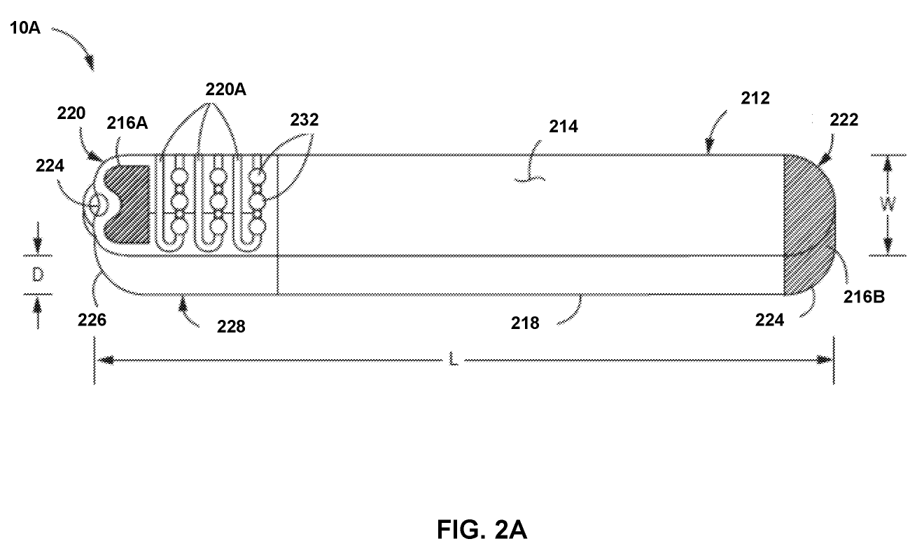

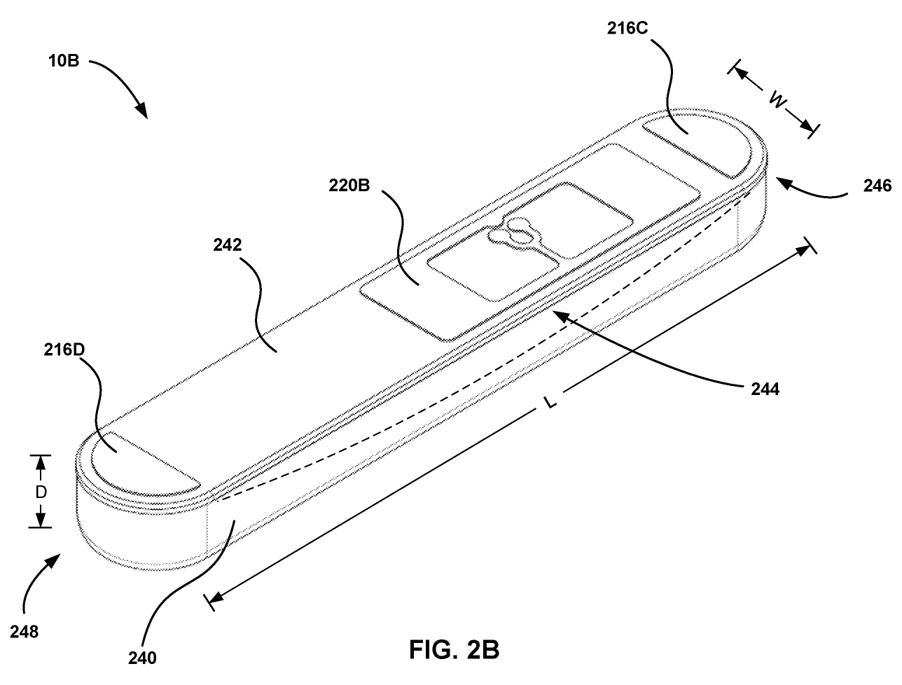

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 212, proximal electrode 216A and distal electrode 216B. Housing 212 may further comprise first major surface 214, second major surface 218, proximal end 220, and distal end 222. Housing 212 encloses electronic circuitry located inside the IMD 10A and protects the circuitry contained therein from body fluids. Housing 212 may be hermetically sealed and configured for subcutaneous implantation. Electrical feedthroughs provide electrical connection of electrodes 216A and 216B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A – in particular, a width W greater than the depth D – is selected to allow IMD 10A to be inserted under the skin of the patient using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 216A and distal electrode 216B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 114 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

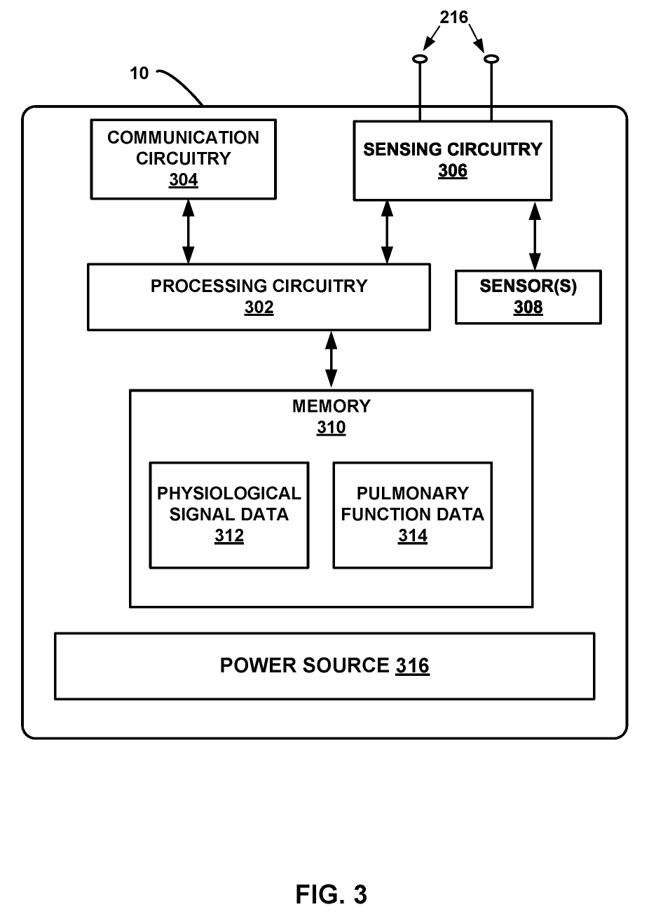

In the example shown in FIG. 2A, once inserted within the patient, the first major surface 214 faces outward, toward the skin of the patient while the second major surface 218 is located opposite the first major surface 214. In addition, in the example shown in FIG. 3A, proximal end 220 and distal end 222 are rounded to reduce discomfort and irritation to surrounding tissue once inserted under the skin of the patient. IMD 10A, including instrument and method for inserting IMD 10A is described, for example, in U.S. Patent Publication No. 2014/0276928, incorporated herein by reference in its entirety.

Proximal electrode 216A is at or proximate to proximal end 220, and distal electrode 216B is at or proximate to distal end 222. Proximal electrode 216A and distal electrode 216B are used to sense one or more physiological signals thoracically outside the ribcage, which may be implanted sub-muscularly or subcutaneously. In some examples, the one or more physiological signals may be stored in a memory of IMD 10A, and data may be transmitted via integrated antenna 220A to another device, which may be another implantable device or an external device, such as external device 212.

In the example shown in FIG. 2A, proximal electrode 216A is at or in close proximity to the proximal end 220 and distal electrode 216B is at or in close proximity to distal end 222. In this example, distal electrode 216B is not limited to a flattened, outward facing surface, but may extend from first major surface 214 around rounded edges 224 and/or end surface 226 and onto the second major surface 218 so that electrode 216B has a three-dimensional curved configuration. In some examples, electrode 216B is an uninsulated portion of a metallic, e.g., titanium, part of housing 212.

In the example shown in FIG. 2A, proximal electrode 216A is located on first major surface 214 and is substantially flat, and outward facing. However, in other examples proximal electrode 216A may utilize the three-dimensional curved configuration of distal electrode 216B, providing a three-dimensional proximal electrode (not shown in this example). Similarly, in other examples distal electrode 216B may utilize a substantially flat, outward facing electrode located on first major surface 214 similar to that shown with respect to proximal electrode 216A.

The various electrode configurations allow for configurations in which proximal electrode 216A and distal electrode 216B are located on both first major surface 214 and second major surface 218. In other configurations, such as that shown in FIG. 2A, only one of proximal electrode 216A and distal electrode 216B is located on both major surfaces 214 and 218, and in still other configurations both proximal electrode 216A and distal electrode 216B are located on one of the first major surface 214 or the second major surface 218 (e.g., proximal electrode 216A is located on first major surface 214 while distal electrode 216B is located on second major surface 218). In another example, IMD 10A may include electrodes on both major surface 214 and 218 at or near the proximal and distal ends of the device, such that a total of four electrodes are included on IMD 10A. Electrodes 216A and 216B may be formed of a plurality of different types of biocompatible conductive material, e.g., stainless steel, titanium, platinum, iridium, or alloys thereof, and may utilize one or more coatings such as titanium nitride or fractal titanium nitride.

In the example shown in FIG. 2A, proximal end 220 includes a header assembly 228 that includes one or more of proximal electrode 216A, integrated antenna 230A, anti-migration projections 232, and/or suture hole 224. Integrated antenna 220A is located on the same major surface (i.e., first major surface 214) as proximal electrode 216A and is also included as part of header assembly 228. Integrated antenna 220A allows IMD 10A to transmit and/or receive data. In other examples, integrated antenna 220A may be formed on the opposite major surface as proximal electrode 216A or may be incorporated within the housing 212 of IMD 10A. In the example shown in FIG. 2A, anti-migration projections 232 are located adjacent to integrated antenna 220A and protrude away from first major surface 214 to prevent longitudinal movement of the device. In the example shown in FIG. 2A, anti-migration projections 232 include a plurality of small bumps or protrusions (e.g., nine small bumps) extending away from first major surface 214. As discussed above, in other examples anti-migration projections 232 may be located on the opposite major surface as proximal electrode 316A and/or integrated antenna 220A. In addition, in the example shown in FIG. 2A, header assembly 228 includes suture hole 224, which provides another means of securing IMD 10A to the patient to prevent movement following insertion. In the example shown, suture hole 224 is located adjacent to proximal electrode 216A. In one example, header assembly 228 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

IMD 10B may include a leadless, subcutaneously-implantable monitoring device, e.g., an ICM. IMD 10B includes housing having a base 240 and an insulative cover 242. Proximal electrode 216C and distal electrode 216D may be formed or placed on an outer surface of cover 242. Various circuitries and components of IMD 10B may be formed or placed on an inner surface of cover 242, or within base 240. In some examples, a battery or other power source of IMD 10B may be included within base 240. In the illustrated example, antenna 220B is formed or placed on the outer surface of cover 242 but may be formed or placed on the inner surface in some examples. In some examples, insulative cover 242 may be positioned over an open base 240 such that base 240 and cover 242 enclose the circuitries and other components and protect them from fluids such as body fluids. The housing including base 240 and insulative cover 242 may be hermetically sealed and configured for subcutaneous implantation.

Circuitries and components may be formed on the inner side of insulative cover 242, such as by using flip-chip technology. Insulative cover 242 may be flipped onto a base 240. When flipped and placed onto base 240, the components of IMD 10B formed on the inner side of insulative cover 242 may be positioned in a gap 244 defined by base 240. Electrodes 216C and 216D and antenna 220B may be electrically connected to circuitry formed on the inner side of insulative cover 242 through one or more vias (not shown) formed through insulative cover 242. Insulative cover 242 may be formed of sapphire (i.e., corundum), glass, parylene, and/or any other suitable insulating material. Base 240 may be formed from titanium or any other suitable material (e.g., a biocompatible material). Electrodes 216C and 216D may be formed from any of stainless steel, titanium, platinum, iridium, or alloys thereof. In addition, electrodes 216C and 216D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 216C and distal electrode 216D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within the patient, outer surface of cover 242 faces outward, toward the skin of the patient. In addition, as shown in FIG. 2B, proximal end 246 and distal end 248 are rounded to reduce discomfort and irritation to surrounding tissue once inserted under the skin of the patient. In addition, edges of IMD 10B may be rounded.

FIG. 3 is a block diagram illustrating an example configuration of an IMD 10 in accordance with one or more techniques described herein. IMD 10 may correspond to either of IMDs 10A and 10B, or another configuration of an IMD. In the illustrated example, IMD 10 includes electrodes 216 (which may correspond to any of electrodes 216A–216D), processing circuitry 302, sensing circuitry 306, sensors 308, communication circuitry 304, power source 316, and memory 310. Although the illustrated example includes two electrodes 316, IMDs including or coupled to more than two electrodes may implement the techniques of this disclosure in some examples. Power source 316 provides operational power for processing circuitry 302, sensing circuitry 306, sensors 308, communication circuitry 304, and memory 310.

Processing circuitry 302 may include fixed function circuitry and/or programmable processing circuitry. Processing circuitry 302 may include any one or more of a microprocessor, a controller, a DSP, an ASIC, a FPGA, a GPU, a TPU, or equivalent discrete or analog logic circuitry. In some examples, processing circuitry 302 may include multiple components, such as any combination of one or more microprocessors, one or more controllers, one or more DSPs, one or more ASICs, or one or more FPGAs, one or more GPUs, one or more TPUs, as well as other discrete or integrated logic circuitry. The functions attributed to processing circuitry 302 herein may be embodied as software, firmware, hardware or any combination thereof.

Sensing circuitry 306 may be coupled to electrodes 216 to sense electrical signals of the heart of patient 4, for example by selecting electrodes 216 and polarity, used to sense one or more physiological signals as controlled by processing circuitry 302. Sensing circuitry 306 may sense the ECG from electrodes 216 in order to facilitate monitoring the electrical activity of the heart. In some examples, sensing circuitry 306 may include one or more filters and amplifiers for filtering and amplifying signals received from electrodes 216 and/or sensors 308. Sensing circuitry 306 and processing circuitry 302 may store physiological signal data 312 and/or pulmonary function data 314 in memory 310. Sensing circuitry 306 may also monitor signals from sensors 308, which may include one or more accelerometers, other vibration or motion sensors, optical sensors, e.g., PPG sensors, or BP sensors, as examples. Sensing circuitry 306 may capture sensor signals from any one of sensors 308 to facilitate monitoring of pulmonary function and patient activity.

Sensing circuitry 306 is configured to sense one or more physiological signals via electrodes 216 and/or sensor(s) and may collect and store physiological signal data based on the one or more sensed physiological signals, e.g., an impedance measurement, an electromyography (EMG) signal, an electrocardiogram (ECG) signal, a heart rate signal, a photoplethysmography (PPG) signal, a tissue oxygen saturation signal, a peripheral oxygen saturation signal, an accelerometer signal, or a pressure signal.

In some examples, processing circuitry 302 and/or other processing circuitry of system 2, e.g., processing circuitry of external device 12, may determine respiration effort and respiration frequency based on the one or more sensed physiological signals. The respiration effort and respiration frequency may be indicative of pulmonary function, e.g., one or more indices of pulmonary function. For example, processing circuitry of system 2, e.g., processing circuitry 302, may determine information corresponding to skeletal muscle activation based on an impedance signal and/or an EMG signal.

The accelerometer signal may be indicative of one or more of a patient posture, a patient activity level, patient heart sounds, or respiration, e.g., respiration frequency. Processing circuitry 302 may determine whether patient 4 performed an exercise correctly based on the patient posture or patient activity level. For example, if system 2 prompted patient 4 to perform a seated breathing exercise, processing circuitry 302 may determine whether patient 4 is seated for the breathing exercise before sensing the one or more physiological signals based on the patient posture. Processing circuitry 302 may additionally or alternatively determine whether patient 4 was seated during the exercise before determining the pulmonary function. In some examples, if the patient posture or activity level meets a corresponding criterion, processing circuitry 302 may control sensing circuitry 306 to sense the one or more physiological signals to determine pulmonary function. Additionally, or alternatively, if the patient posture or activity level meets the corresponding criterion, processing circuitry 302 may determine to prompt patient 4 to perform an exercise.

Processing circuitry 302 may track patient breathing based on one or more of the physiological signals. As examples, the impedance signal, the accelerometer signal, and/or the ECG signal may be indicative of a pulmonary volume of patient 4. For example, based on R-wave amplitudes of the ECG signal, processing circuitry 302 may determine the pulmonary volume of patient 4. Additionally, or alternatively, the impedance signal and/or the ECG signal may be indicative of airflow of patient 4. In some examples, the EMG signal may be indicative of a force of an exhaled patient breath.

In some examples, the one or more physiological signals may additionally or alternatively be indicative of overall patient health. For example, processing circuitry 302 may determine a resting heart rate and/or an average heart rate based on the heart rate signal. The value of the resting heart rate and/or the average heart rate may be indicative of an overall patient health. As another example, the tissue oxygen saturation signal and/or the peripheral oxygen saturation signal may be indicative of the overall patient health.

Based on the one or more sensed physiological signals, system 2, e.g., processing circuitry 302 of IMD 10 and/or processing circuitry of external device 12, may determine a pulmonary function of patient 4. In some examples, IMD 10 senses the one or more physiological signals when patient 4 is performing an exercise, e.g., a breathing or walking exercise. In some examples, external device 12 prompts patient 4 to perform the exercise according to a periodic schedule. In some examples, one or more elements of system 2 may assess a quality associated with the one or more physiological signals. For example, processing circuitry 302 may determine one or more of whether patient 4 was in a seated position during an exercise based on accelerometer data or a patient effort associated with the exercise based on one or more of the physiological signals, patient input, and/or one or more previous signals. If patient 4 was not in a seated position during the exercise or if the patient effort falls below a threshold, processing circuitry 302 may determine the quality associated with the one or more physiological signals does not meet a quality criterion. Processing circuitry 302 may determine not to determine the pulmonary function and may control communication circuitry 304 prompt patient 4 to redo the exercise at a later time, e.g., at a later time with a window defined by the periodic schedule.

In some examples, system 2 additionally or alternatively determines the pulmonary function of patient 4 based on one or more physiological signals sensed continuously, e.g., hourly, and not corresponding to when patient 4 performs the exercise. In other words, processing circuitry 302 and/or processing circuitry of external device 12 may determine pulmonary function “in the background.”

Communication circuitry 304 may include any suitable hardware, firmware, software, or any combination thereof for communicating with another device, such as external device 12, another networked computing device, or another IMD or sensor. Under the control of processing circuitry 302, communication circuitry 304 may receive downlink telemetry from, as well as send uplink telemetry to external device 12. In addition, processing circuitry 302 may communicate with a networked computing device via an external device (e.g., external device 12) and a computer network, such as the Medtronic CareLink™ Network. Communication circuitry 304 may be configured to transmit and/or receive signals via inductive coupling, electromagnetic coupling, Near Field Communication (NFC), Radio Frequency (RF) communication, Bluetooth, Wi-Fi, or other proprietary or non-proprietary wireless communication schemes.

Memory 310 may be configured to store information within IMD 10 during operation. Memory 310, in some examples, is described as a computer-readable storage medium. In some examples, memory 310 is a temporary memory, meaning that a primary purpose of memory 310 is not long-term storage. Memory 310, in some examples, is described as a volatile memory, meaning that memory 310 does not maintain stored contents when the computer is turned off. Examples of volatile memories include RAM, dynamic RAM (DRAM), static RAM (SRAM), and other forms of volatile memories known in the art. In some examples, memory 310 is used to store program instructions for execution by processing circuitry 302. Memory 310, in one example, is used by software or applications running on IMD 10 to temporarily store information during program execution.

Memory 310, in some examples, also includes one or more non-transitory computer-readable storage media. Memory 310 may be configured to store larger amounts of information than volatile memory. Memory 310 may further be configured for long-term storage of information. In some examples, memory 310 includes non-volatile storage elements. Examples of such non-volatile storage elements include magnetic hard discs, optical discs, floppy discs, flash memories, or forms of electrically programmable memories (EPROM) or EEPROM memories.

In some examples, processing circuitry 302 may process physiological signal data 312 to determine a pulmonary function of patient 4. In some examples, processing circuitry 302 may control communication circuitry 304 to transmit physiological signal data 312 or metric data to another device, e.g., to external device 12, for analysis to determine pulmonary function according to the techniques of this disclosure. In some examples, IMD 10 may store trends in pulmonary function in pulmonary function data 314. Memory 310 may be configured to store patient data, e.g., historical patient COPD data, medication schedule, and/or comorbid conditions. Memory 310 may be configured to store user input, e.g., user input regarding an exercise, symptoms, and/or medication information.

Processing circuitry 302 may also use activity and/or posture signal information, e.g., determined via an accelerometer signal, to determine an activity level and/or posture associated with collection of the one or more physiological signals, e.g., one or more physiological signals sensed while patient 4 performed an exercise according to a periodic schedule or one or more physiological signals sensed continuously. In some examples, processing circuitry 302 may determine whether to use the one or more physiological signals to determine a pulmonary function based on the activity and/or posture information. As an example, if system 2 instructed patient 4 to perform a breathing exercise in a seated position but posture information determined via the accelerometer signal indicates patient 4 was standing or laying down during the breathing test, processing circuitry 302 may determine not to determine pulmonary function based on the one or more physiological signals. In some examples, processing circuitry 302 may initiate sensing one or more physiological signals in response to patient activity and/or posture meeting one or more criteria. For example, if an activity level of patient 4 drops below a threshold activity level, processing circuitry 302 may determine to sense one or more physiological signals to determine pulmonary function.

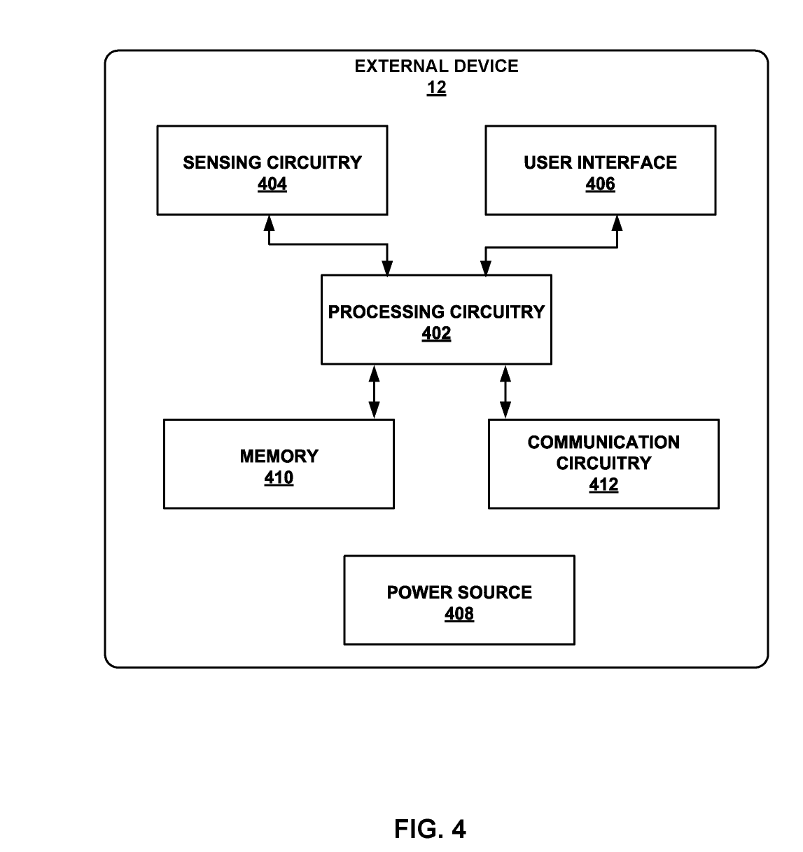

FIG. 4 is a block diagram illustrating an example configuration of an external device 12 that operates in accordance with one or more techniques of this disclosure. External device 12 is configured to communicate with any IMD (e.g., IMD 10) described herein, in accordance with one or more techniques of this disclosure. In the example of FIG. 4, external device 12 includes processing circuitry 402, communication circuitry 412, user interface 406, sensing circuitry 404, power source 408, and memory 410.

Processing circuitry 402, in one example, may include one or more processors that are configured to implement functionality and/or process instructions for execution within external device 12. For example, processing circuitry 402 may be capable of processing instructions stored in memory 410. Processing circuitry 402 may include, for example, microprocessors, DSPs, ASICs, FPGAs, GPUs, TPUs, or equivalent discrete or integrated logic circuitry, or a combination of any of the foregoing devices or circuitry. Accordingly, processing circuitry 402 may include any suitable structure, whether in hardware, software, firmware, or any combination thereof, to perform the functions ascribed herein to processing circuitry 402.

Communication circuitry 412 may include any suitable hardware, firmware, software or any combination thereof for communicating with another device, such as IMD 10. Under the control of processing circuitry 402, communication circuitry 412 may receive downlink telemetry from, as well as send uplink telemetry to, IMD 10, or another device.

A user, such as a clinician or patient 4, may interact with external device 12 through user interface 406. User interface 406 includes a display (not shown), such as an LCD or LED display or other type of screen, with which processing circuitry 402 may present information related to IMD 10 (e.g., a detection of a cardiac episode). In addition, user interface 406 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 402 of external device 12 and provide input, e.g., symptoms, medication timing, e.g., before and/or after bronchodilation treatment, and/or testing quality assessments. In other examples, user interface 406 also includes audio circuitry for providing audible notifications, instructions or other sounds to patient 4, receiving voice commands from patient 4, or both. Memory 410 may include instructions for operating user interface 406 and for managing power source 408.

User interface 406 may be configured to display prompts to patient 4 to perform an exercise, e.g., a walking exercise and/or a breathing exercise, according to a periodic schedule. User interface 406 may be configured to display instructions regarding the patient exercise and/or feedback for the performance of patient 4. For example, user interface 406 may instruct patient 4 to perform a 6-minute walking test across a flat surface. As another example, user interface may instruct patient 4 to take a deep breath in and out. In some examples, patient 4 may provide input to indicate patient 4 has started the exercise. In some examples, patient 4 may provide input to indicate conclusion of the exercise.

User interface 406 may be configured to display output indicative of pulmonary function for user review. For example, user interface 406 may be configured to display a trend in pulmonary function, a suggested adjustment in medication and/or therapy, an indication to use a rescue inhaler, and/or a suggestion to schedule an in-person clinic visit, to the user, e.g., the clinician, a caregiver, and/or patient 4. In some examples, based on the nature of the output indicative of pulmonary function, processing circuitry 402 may determine whether to provide the output to the clinician, the caregiver, patient 4, or a combination thereof. For example, processing circuitry 402 may determine to output a trend in pulmonary function for clinician review. Processing circuitry 402 may determine to output an indication to use a rescue inhaler to patient 4 and/or the caregiver. In some examples, processing circuitry 402 may determine to send data to a chart of patient 4 and not generate a notification. In some examples, the output may include one or more graphical representations, e.g., flow plots or flow volume plots.

In some examples, e.g., in examples in which external device 12 comprises a patient computing device, such as a patient smartphone or smart watch, external device 12 may include sensing circuitry 404. Sensing circuitry 404 may be configured to sense one or more of the one or more physiological signals system 2 uses to determine pulmonary function. As an example, when external device 12 instructs patient 4 to perform a deep breathing example and when external device 12 comprises a patient smartphone, sensing circuitry 404 may sense, via a microphone of the patient smartphone, a breathing signal of patient 4. In some examples, user interface 406 may display an instruction to patient 4 to breathe into the microphone of computing device 12. As another example, e.g., in an example in which external device 12 comprises a smart watch, sensing circuitry 404 may sense one or more of the one or more physiological signals, such as a patient heart rate, a patient tissue oxygen saturation, etc. Processing circuitry 402 and/or processing circuitry 302 may determine, based on the one or more physiological signals, the pulmonary function of patient 4.

Power source 408 is configured to deliver operating power to the components of external device 12. Power source 408 may include a battery and a power generation circuit to produce the operating power. In some examples, the battery is rechargeable to allow extended operation. Recharging may be accomplished by electrically coupling power source 408 to a cradle or plug that is connected to an alternating current (AC) outlet. In addition, recharging may be accomplished through proximal inductive interaction between an external charger and an inductive charging coil within external device 10. In other examples, traditional batteries (e.g., nickel cadmium or lithium ion batteries) may be used. In addition, external device 12 may be directly coupled to an alternating current outlet to operate.

Memory 410 may be configured to store information within external device 12 during operation. In some examples, memory 410 may be referred to as a storage device and include computer-readable instructions that, when executed by processing circuitry 402, cause external device 12 and processing circuitry 402 to perform various functions attributed to external device 12 and processing circuitry 402 herein. Memory 410 may include any volatile, non-volatile, magnetic, optical, or electrical media, such as RAM, DRAM, SRAM, magnetic discs, optical discs, flash memories, ROM, NVRAM, EPROM, EEPROM, flash memory, or any other digital media. Memory 410 may also store data generated by sensing circuitry 404 of IMD 10, such as signals corresponding to indications of detections.

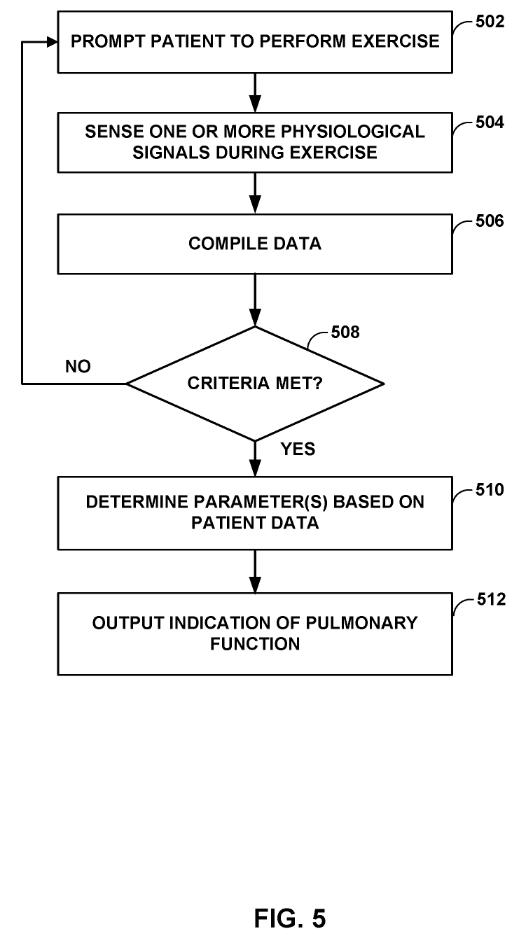

FIG. 5 is a flow diagram illustrating an example operation for determining a pulmonary function of a patient based on one or more sensed physiological signals meeting one or more corresponding criteria, in accordance with one or more techniques of this disclosure. Although described as being performed primarily by processing circuitry 402 of external device 12, the example operation of FIG. 5 may additionally or alternatively be performed by any processing circuitry of system 2, e.g., processing circuitry 302 of IMD 10 or processing circuitry of a computing system of system 2 (not depicted).

Processing circuitry 402 prompts a patient, e.g., patient 4, to perform an exercise (502). In some examples, processing circuitry 402 prompts, via user interface 406, patient 4 to perform the exercise according to a periodic schedule, e.g., multiple times a day, daily, weekly, or monthly. In some examples, processing circuitry 402 prompts patient 4 to perform the exercise in response to one or more environmental and/or patient criteria being met. For example, processing circuitry 402 may determine to prompt patient 4 to perform the exercise in response to an environment of patient 4 meeting a smog criterion. In some examples, processing circuitry 402 may determine to prompt patient 4 to perform the exercise based on a lung-clearing schedule. Processing circuitry 402 may determine to prompt patient 4 to perform the exercise based on user input, one or more sensed physiological signals indicative of a change in pulmonary function, or a change in patient medication, e.g., a change in medication prescription and/or in response to a determination that patient 4 used a rescue inhaler. In some examples, the rescue inhaler may be configured to wirelessly communicate with IMD 10 and/or external device 12. The rescue inhaler may communicate use of the rescue inhaler by patient 4. In some examples, patient 4 or another user may provide user input indicative of the user of the rescue inhaler. In some examples, processing circuitry 402 may prompt patient 4 to perform the exercise in order to assess an efficacy associated with the change in medication prescription. Processing circuitry 402 may determine to prompt patient 4 to perform the exercise based on a patient activity level or patient posture meeting a corresponding criterion.

Processing circuitry 402 controls sensing circuitry 306 of IMD 10 and/or sensing circuitry 404 of external device 12 to sense one or more physiological signals of patient 4 while patient 4 performs the exercise (504). The one or more physiological signals may include one or more of an impedance measurement, an electromyography (EMG) signal, an electrocardiogram (ECG) signal, a heart rate signal, a photoplethysmography (PPG) signal, a tissue oxygen saturation signal, a peripheral oxygen saturation signal, an accelerometer signal, or a pressure signal. In some examples, one or more of the one or more physiological signals may be signals sensed from lung tissue. Processing circuitry 402 compiles data (506). In some examples, the data comprises one or more of current physiological signal data, previous physiological signal data, user input, patient medication data, patient history data, and/or environmental data. Processing circuitry 402 determines whether the data meets one or more corresponding criteria (508). For example, processing circuitry 402 may determine whether a quality associated with one or more of the one or more physiological signals meets a corresponding quality threshold. For example, processing circuitry 402 may determine whether a patient effort associated with one or more of the one or more physiological signals meets a patient effort threshold. To determine the patient effort, processing circuitry 402 may compare one or more signal amplitudes, e.g., EMG signal amplitude, to a threshold and/or one or more previous signal amplitudes sensed during a previous performance of the exercise. In some examples, the threshold may be patient specific. In some examples, the threshold changes as the COPD progression of patient 4 changes. Processing circuitry 402 may additionally or alternatively compare a duration of the exercise to a threshold and/or one or more previous durations. The threshold may be patient specific and/or may change as the COPD progression of patient 4 changes. As another example, processing circuitry 402 may determine whether a patient posture or activity level meets a corresponding criterion.

If the one or more corresponding criteria are not met (“NO” of 508), processing circuitry 402 determines to prompt patient 4 to perform the exercise again at a later time, e.g., at a later time within a window defined by the periodic schedule (502). If the one or more corresponding criteria are met (“YES” of 508), processing circuitry 402 determines one or more parameters indicative of pulmonary function based on the one or more physiological signals and/or other patient data (510). The one or more parameters may include one or more indices of pulmonary function, such as FEV1, FVC, FEV1 to FVC ratio, FEF, PEF, and/or an overall health of patient 4. Based on the one or more parameters, processing circuitry 402 controls user interface 406 to output an indication of pulmonary function for user review, e.g., patient 4, caregiver, and/or clinician review (512). The output may include one or more of the pulmonary function, e.g., the one or more indices of pulmonary function, a trend in pulmonary function, e.g., a trend in one or more indices of pulmonary function, an indication to adjust a medication, an indication of an efficacy associated with medication, and/or an indication to use a rescue inhaler. In some examples, processing circuitry 402 may determine to send data to a chart of patient 4 for clinician review and not generate a notification. In some examples, the output may include one or more graphical representations, e.g., flow plots or flow volume plots.

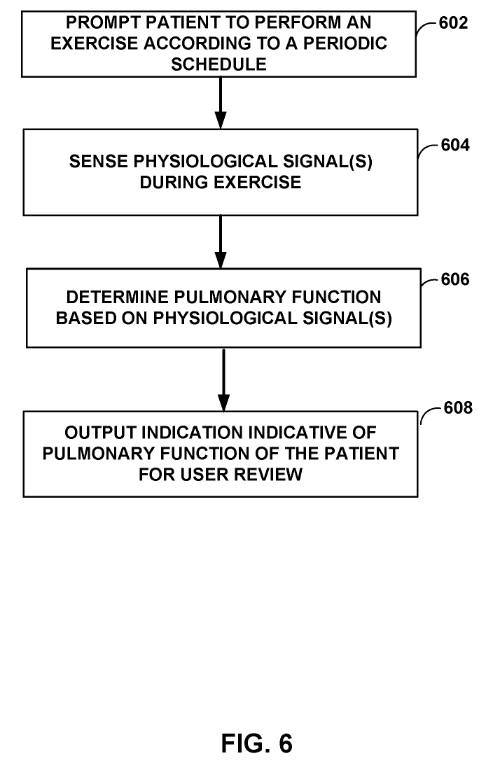

FIG. 6 is a flow diagram illustrating an example operation for determining a pulmonary function of a patient based on signals sensed while the patient performed an exercise, in accordance with one or more techniques of this disclosure. Although described as being performed primarily by processing circuitry 402 of external device 12, the example operation of FIG. 6 may additionally or alternatively be performed by any processing circuitry of system 2, e.g., processing circuitry 302 of IMD 10 or processing circuitry of a computing system of system 2 (not depicted).

Processing circuitry 402 prompts, via user interface 406 of external device 12, a patient, e.g., patient 4, to perform an exercise, e.g., a breathing exercise and/or a walking exercise, according to a periodic schedule, e.g., a daily schedule (602). In some examples, the periodic schedule is based on a patient schedule. For example, processing circuitry 402 and/or the user may set the periodic schedule such that patient 4 is usually at home when processing circuitry 402 prompts patient 4 to perform the exercise. In some examples, the periodic schedule is based on a patient medication schedule. For example, processing circuitry 402 may determine to prompt the user to perform the exercise a predetermined amount of time before or after taking a medication. In some examples, processing circuitry 402 may otherwise account for medication timing when determining pulmonary function. In some examples, the periodic schedule may change over time. As an example, COPD progresses, processing circuitry 402 may determine to prompt patient 4 to perform the exercise more or less frequently.

In some examples, the periodic schedule may designate a window of time, e.g., a one hour window, in which processing circuitry 402 prompts patient 4 to perform the exercise. Processing circuitry 402 may determine a time within the window at which to prompt patient 4 to perform the exercise. For example, processing circuitry 402 may prompt patient 4 to perform the exercise at a time within the window at which patient 4 is at rest.

Processing circuitry 402 controls sensing circuitry 306 of IMD 10 and/or sensing circuitry 404 of external device 12 to sense one or more physiological signals of patient 4 (604). The one or more physiological signals may include one or more of an impedance measurement, an EMG signal, an ECG signal, a heart rate signal, a PPG signal, a tissue oxygen saturation signal, a peripheral oxygen saturation signal, an accelerometer signal, or a pressure signal.

Processing circuitry 402 determines a pulmonary function of patient 4, e.g., one or more indices of pulmonary function, based on the one or more physiological signals (606). In some examples, processing circuitry 402 determines whether a quality associated with the one or more physiological signals and/or the performance of the exercise meets a corresponding quality criterion. For example, processing circuitry 402 may determine whether a patient effort associated with the one or more physiological signals and/or the performance of the exercise meets an effort criterion. As another example, processing circuitry 402 may determine conditions in which patient 4 performed the exercise. For example, if patient 4 performed the exercise in an environment with low air quality, e.g., smog, processing circuitry 402 may determine to adjust a determination of pulmonary function based on the environment and/or may determine not to determine the pulmonary function of patient 4 using the one or more physiological signals. In some examples, processing circuitry 402 may additionally continuously sense the one or more physiological signals and determine pulmonary function of the patient based on the continuously sensed one or more physiological signals.

Processing circuitry 402 controls user interface 406 to output an indication indicative of the pulmonary function of patient 4 (608). The output may include one or more of the pulmonary function, e.g., the one or more indices of pulmonary function, such as FEV1, FVC, FEV1 to FVC ratio, FEF, and/or PEF, a trend in pulmonary function, e.g., a trend in one or more indices of pulmonary function, an indication to adjust a medication, an efficacy associated with the medication, and/or an indication to use a rescue inhaler. In some examples, the output may include one or more graphical representations, e.g., flow plots or flow volume plots.

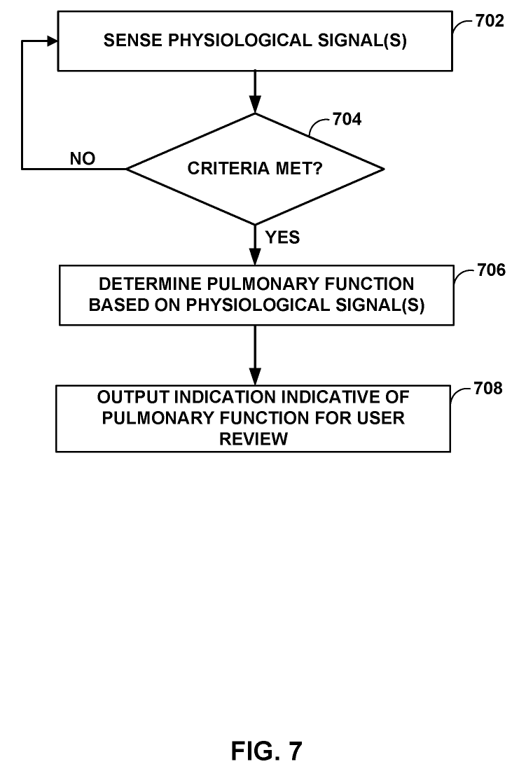

FIG. 7 is a flow diagram illustrating an example operation for determining a pulmonary function of a patient based on signals sensed according to a periodic schedule, in accordance with one or more techniques of this disclosure. Although described as being performed primarily by processing circuitry 402 of external device 12, the example operation of FIG. 7 may additionally or alternatively be performed by any processing circuitry of system 2, e.g., processing circuitry 302 of IMD 10 or processing circuitry of a computing system of system 2 (not depicted).

Sensing circuitry 306 of IMD 10 senses one or more physiological signals of patient 4 (702). The one or more physiological signals may include one or more of an impedance measurement, an EMG signal, an ECG signal, a heart rate signal, a PPG signal, a tissue oxygen saturation signal, a peripheral oxygen saturation signal, an accelerometer signal, or a pressure signal. In some examples, sensing circuitry 306 senses the one or more physiological signals continuously, e.g., on a periodic schedule, such as hourly. In some examples, processing circuitry 402 determines to control sensing circuitry 306 to sense the one or more physiological signals in response to one or more environmental and/or patient criteria being met. For example, processing circuitry 402 may determine to control sensing circuitry 306 to sense the one or more physiological signals in response to an environment of patient 4 meeting a smog criterion. Processing circuitry 402 may determine to control sensing circuitry 306 to sense the one or more physiological signals based on user input, one or more sensed physiological signals indicative of a change in pulmonary function, or a change in patient medication. Processing circuitry 402 may determine to control sensing circuitry 306 to sense the one or more physiological signals based on a patient activity level or patient posture meeting a corresponding criterion. As an example, if processing circuitry 402 determines the patient activity level meets an activity threshold, processing circuitry 402 may determine to control sensing circuitry 306 to sense the one or more physiological signals.

In some examples, processing circuitry 402 determines whether the one or more physiological signals meet one or more criteria (704). As an example, processing circuitry 402 may determine whether a quality associated with the one or more physiological signals meets a quality criterion. For example, processing circuitry 402 may determine whether a patient posture, patient activity level, or environmental condition associated with the one or more physiological signals meets a corresponding quality criterion. In some examples, processing circuitry 402 determines whether a signal to noise ratio (SNR), signal amplitude, or another indicator of signal quality meets a corresponding quality criterion.

If processing circuitry 402 determines the one or more physiological signals do not meet the one or more criteria (“NO” of 704), processing circuitry 402 determines to continue sensing (702). In some examples, processing circuitry 402 may determine not to use the one or more sensed physiological signals to determine pulmonary function. If processing circuitry 402 determines the one or more physiological signals meet the one or more criteria (“YES” of 704), processing circuitry determines pulmonary function based on the one or more physiological signals (706). In some examples, determining pulmonary function includes determining one or more indices of pulmonary function, such as FEV1, FVC, FEV1 to FVC ratio, FEF, and/or PEF. Processing circuitry 402 controls user interface 406 to output an indication indicative of pulmonary function for user review, e.g., patient 4, clinician, and/or caregiver review (708). The output indicative of pulmonary function may include trends in pulmonary function, e.g., trends in indices of pulmonary function, the pulmonary function, e.g., one or more indices of pulmonary function, overall health, an indication to adjust a medication or therapy based on pulmonary function, an efficacy associated with the medication, e.g., an efficacy associated with a change in medication, and/or an indication to use a rescue inhaler. In some examples, processing circuitry 402 may determine to send data to a chart of patient 4 for clinician review and not generate a notification. In some examples, the output may include one or more graphical representations, e.g., flow plots or flow volume plots.

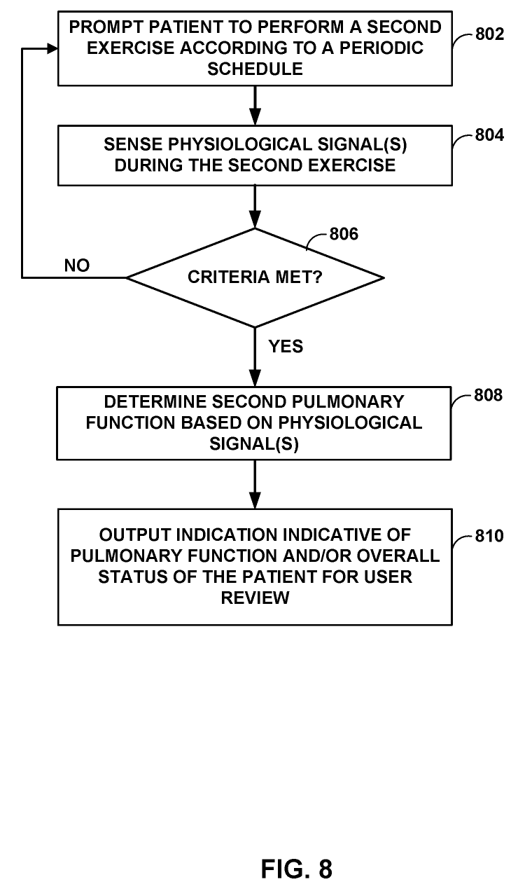

FIG. 8 is a flow diagram illustrating an example operation for determining an overall pulmonary function of a patient based on signals sensed while the patient performed a second exercise, in accordance with one or more techniques of this disclosure. Although described as being performed primarily by processing circuitry 402 of external device 12, the example operation of FIG. 8 may additionally or alternatively be performed by any processing circuitry of system 2, e.g., processing circuitry 302 of IMD 10 or processing circuitry of a computing system of system 2 (not depicted). In some examples, the example operation of FIG. 8 is performed in addition to the example operation of FIGS. 5, 6, and/or 7.

Processing circuitry 402 prompts patient 4 to perform a second exercise according to a periodic schedule (802). In examples in which the example operation of FIG. 8 is performed in addition to the example operation of FIGS. 5 or 6, processing circuitry 402 may prompt patient 4 to perform the second exercise after a first exercise corresponding to the example operation of FIGS. 5 or 6. In some examples, the example operation of FIG. 8 is offset from the example operation of FIG. 5 or FIG. 6. In some examples, the example operation of FIG. 8 is performed less frequently than the example operation of FIGS. 5 or 6.

The second exercise may comprise an exercise that was not previously performed by patient 4. For example, if the example operation of FIGS. 5 or 6 included prompting patient 4 to perform a seated breathing test, the example operation of FIG. 8 may include prompting patient 4 to perform a walking test, such as a 6-minute hallway walking test. Sensing circuitry 306 of IMD 10, and, in some examples, sensing circuitry 404 of external device 12, sense one or more physiological signals of patient 4 while patient 4 performs the second exercise (804). The one or more physiological signals may include one or more of an impedance measurement, an EMG signal, an ECG signal, a heart rate signal, a PPG signal, a tissue oxygen saturation signal, a peripheral oxygen saturation signal, an accelerometer signal, or a pressure signal. Processing circuitry 402 determines whether the one or more physiological signals meet one or more criteria (806). As an example, processing circuitry 402 may determine whether a quality associated with the one or more physiological signals meets a quality criterion. For example, processing circuitry 402 may determine whether a patient effort, a patient posture, a patient activity level, or an environmental condition associated with the one or more physiological signals meets a corresponding quality criterion. In some examples, processing circuitry 402 determines whether a SNR, a signal amplitude, or another indicator of signal quality meets a corresponding quality criterion.

If processing circuitry 402 determines the one or more criteria are not met (“NO” of 806), processing circuitry 402 determines to prompt patient 4 to perform the second exercise again (802). In some examples, processing circuitry 402 determines to prompt patient 4 to perform the second exercise again at a later time, e.g., later in the day. If processing circuitry 402 determines the one or more criteria are met (“YES” of 806), processing circuitry 402 determines a second pulmonary function, e.g., a second set of one or more indices of pulmonary function, based on the one or more physiological signals and/or an overall health status of patient 4 (808). In some examples, processing circuitry 402 determines different indices of pulmonary function based on the one or more physiological signals sensed while patient 4 performed the second exercise than the indices based on the one or more physiological signals sensed while patient 4 performed the first exercise, e.g., the exercise of FIGS. 5 or 6.

Processing circuitry 402 controls user interface 406 to output an indication indicative of the pulmonary function and/or an overall health status of patient 4 for user review, e.g., patient 4, clinician, and/or caregiver review (810). The output indicative of pulmonary function may include trends in pulmonary function, e.g., trends in indices of pulmonary function, the pulmonary function, e.g., one or more indices of pulmonary function, overall health, an indication to adjust a medication or therapy based on pulmonary function, an efficacy associated with the medication, e.g., an efficacy associated with the change in medication, and/or an indication to use a rescue inhaler. In some examples, processing circuitry 402 determines an overall pulmonary function based on the first pulmonary function, e.g., the pulmonary function determined via the example operation of FIGS. 5 or 6, and the second pulmonary function, e.g., the pulmonary function determined via the example operation of FIG. 8.

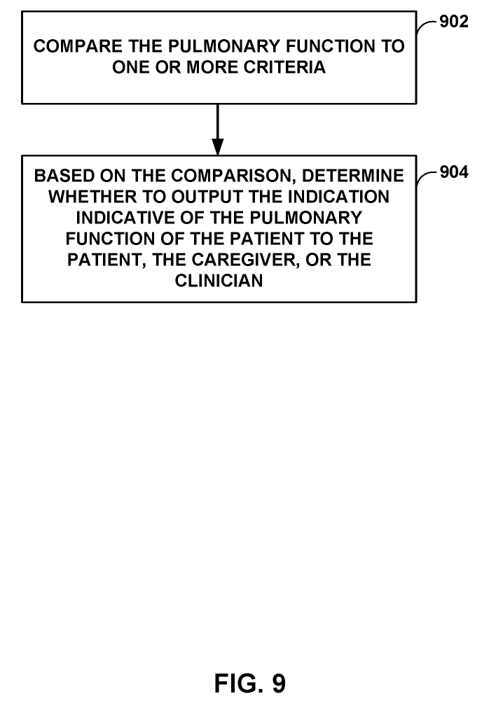

FIG. 9 is a flow diagram illustrating an example operation for determining whether to output an indication indicative of the pulmonary function to the patient, a clinician, a caregiver, or a combination thereof, in accordance with one or more techniques of this disclosure. Although described as being performed primarily by processing circuitry 402 of external device 12, the example operation of FIG. 9 may additionally or alternatively be performed by any processing circuitry of system 2, e.g., processing circuitry 302 of IMD 10 or processing circuitry of a computing system of system 2 (not depicted).

Processing circuitry 402 compares the pulmonary function, e.g., the pulmonary function determined via the example operation of any of FIGS. 5, 6, 7, or 8, to one or more criteria (902). Based on the comparison, processing circuitry 402 determines whether to output the indication indicative of the pulmonary function of patient 4 to patient 4, the caregiver, or the clinician (904).

The one or more criteria may include an urgency criterion. For example, if the output indicative of the pulmonary function comprises an indication to use a rescue inhaler, processing circuitry 402 may determine to output the indication to patient 4 and/or the caregiver. Processing circuitry 402 may determine not to output the indication to the clinician in this example since only patient 4 or the caregiver may be able to administer the rescue inhaler while patient 4 is at home or otherwise not located at the clinic. In examples in which the pulmonary function falls below an urgency criterion, processing circuitry 402 may determine to send data to a chart of patient 4 for clinician review and not generate a notification.

In some examples, the one or more criteria may include a criterion indicative of which user(s) can confirm indications to make changes. For example, if the output indicative of the pulmonary function comprises an indication to adjust a medication, processing circuitry 402 may determine to output the indication to the clinician or another user able to adjust a medication of patient 4. In some examples, patient 4 may not be able to adjust a medication prescription. Therefore, processing circuitry 402 may determine to output the indication to adjust the medication to the clinician and not to the patient.

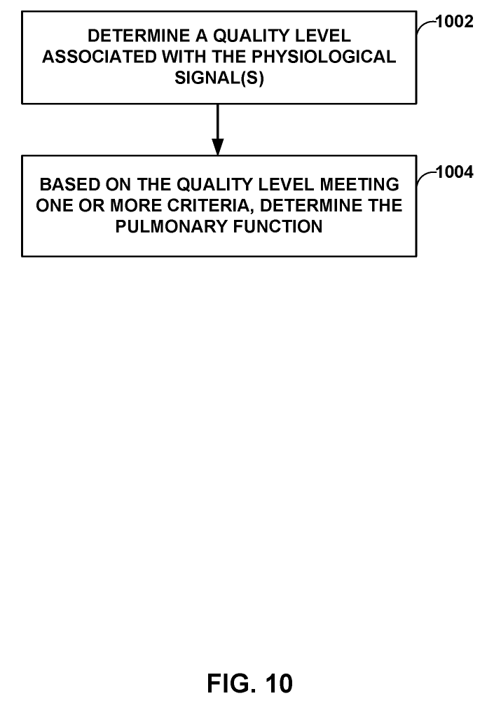

FIG. 10 is a flow diagram illustrating an example operation for determining whether to determine pulmonary function based on a quality level of the one or more physiological signals, in accordance with one or more techniques of this disclosure. Although described as being performed primarily by processing circuitry 402 of external device 12, the example operation of FIG. 10 may additionally or alternatively be performed by any processing circuitry of system 2, e.g., processing circuitry 302 of IMD 10 or processing circuitry of a computing system of system 2 (not depicted). In some examples, processing circuitry 402 performs the example operation of FIG. 10 in addition to the example operation of FIG. 6.

Processing circuitry 402 determines a quality level associated with the one or more physiological signals (1002). The one or more physiological signals may include one or more of an impedance measurement, an EMG signal, an ECG signal, a heart rate signal, a PPG signal, a tissue oxygen saturation signal, a peripheral oxygen saturation signal, an accelerometer signal, or a pressure signal. Based on the quality level meeting one or more criteria, processing circuitry 402 determines the pulmonary function based on the one or more physiological signals (1004). For example, processing circuitry 402 may determine whether a patient effort, a patient posture, a patient activity level, or an environmental condition associated with the one or more physiological signals meets a corresponding quality criterion. To determine whether the patient effort associated with the one or more physiological signals meets the corresponding quality criterion, processing circuitry 402 may determine whether the patient effort meets a threshold level. Processing circuitry 402 may determine the patient effort based on one or more of the one or more physiological signals. In some examples, the threshold level may be configurable. As an example, the clinician may select a threshold level. In some examples, the threshold level may be patient specific. In such examples, processing circuitry 402 and/or the clinician may determine a threshold level for the patient based on the one or more physiological signals or other input, such as patient input or historical patient data. In some examples, processing circuitry 402 determines whether a SNR, a signal amplitude, or another indicator of signal quality meets a corresponding quality criterion.

The following is a non-limiting list of examples that are in accordance with one or more techniques of this disclosure.

Example 1. A system comprising: an implantable medical device (IMD) comprising sensing circuitry configured to sense one or more physiological signals of a patient; and processing circuitry configured to: prompt, via a user interface, the patient to perform an exercise according to a periodic schedule; control the sensing circuitry to sense the one or more physiological signals of the patient while the patient performs the exercise; based on the one or more physiological signals sensed while the patient performs the exercise, determine a pulmonary function of the patient; and output an indication indicative of the pulmonary function of the patient for user review.

Example 2. The system of example 1, wherein the exercise comprises a breathing exercise.

Example 3. The system of examples 1 or 2, wherein the exercise is a first exercise and the pulmonary function of the patient comprises a first pulmonary function, and wherein the processing circuitry is further configured to: prompt, via the user interface, the patient to perform a second exercise according to the periodic schedule; control the sensing circuitry to sense the one or more physiological signals of the patient of the patient while the patient performs the second exercise; based on the one or more physiological signals sensed while the patient performs the second exercise, determine a second pulmonary function of the patient; determine, based on the first pulmonary function and the second pulmonary function of the patient, an overall pulmonary function of the patient; and output an indication indicative of the overall pulmonary function of the patient for user review.

Example 4. The system of example 3, wherein the first exercise comprises a deep breathing exercise, and wherein the second exercise comprises a walking exercise.

Example 5. The system of any of examples 1–4, wherein to determine the pulmonary function of the patient, the processing circuitry is configured to determine one or more indices of pulmonary function, wherein the one or more indices of pulmonary function comprise one or more of: a forced expiratory volume in one second (FEV1); a forced vital capacity (FVC); an FEV1 to FVC ratio; a forced mid-expiratory flow (FEF 25-75); or a peak expiratory flow (PEF).

Example 6. The system of any of examples 1–5, wherein the processing circuitry is further configured to: determine the pulmonary function of the patient based on the one or more physiological signals sensed continuously according to a periodic schedule.

Example 7. The system of any of examples 1–6, wherein the user comprises one or more of: a clinician; a caregiver; or the patient.

Example 8. The system of example 7, wherein the processing circuitry is further configured to: compare the pulmonary function of the patient to one or more corresponding criteria; and based on the comparison, determine whether to output the indication indicative of the pulmonary function of the patient to the patient, the caregiver, or the clinician.

Example 9. The system of any of examples 1–8, wherein the output indicative of the pulmonary function of the patient comprises one or more of: a trend in pulmonary function; a suggestion to adjust a patient treatment; or an indication to use a rescue inhaler.

Example 10. The system of any of examples 1–9, wherein the processing circuitry is configured to: adjust the periodic schedule based on one or more of: the pulmonary function of the patient; an overall health of the patient; or one or more environmental factors.

Example 11. The system of any of examples 1–10, wherein the processing circuitry is further configured to: determine a quality level associated with one or more of the one or more physiological signals; and based on the quality level of the one or more physiological signals meeting one or more criteria, determine the pulmonary function of the patient.

Example 12. The system of any of examples 1–11, wherein the one or more physiological signals include one or more of: an impedance measurement; an electromyography (EMG) signal; an electrocardiogram (ECG) signal; a heart rate signal; a photoplethysmography (PPG) signal; a tissue oxygen saturation signal; a peripheral oxygen saturation signal; an accelerometer signal; or a pressure signal.

Example 13. The system of any of examples 1–12, wherein the indication indicative of the pulmonary function of the patient comprises an indication of a chronic obstructive pulmonary disease (COPD) status of the patient.

Example 14. The system of any of examples 1–13, wherein the user interface comprises a user interface of a user device.

Example 15. The system of any one or more of examples 1–14, wherein the IMD comprises a housing configured for subcutaneous implantation, the housing having a length, a width, and a depth, wherein the length is greater than the width, and the width is greater than the depth, and the length is within a range from 40 millimeters (mm) to 60 mm, and wherein the IMD comprises a plurality of sensors on the housing configured to sense the one or more physiological signals.

Example 16. A method comprising: prompting, by processing circuitry of a medical device system comprising an implantable medical device (IMD) and via a user interface, a patient to perform an exercise according to a periodic schedule; controlling, by the processing circuitry, sensing circuitry of the IMD to sense one or more physiological signals of the patient while the patient performs the exercise; based on the one or more physiological signals sensed while the patient performs the exercise, determining, by the processing circuitry, a pulmonary function of the patient; and outputting, by the processing circuitry, an indication indicative of the pulmonary function of the patient for user review.

Example 17. The method of example 16, wherein the exercise comprises a breathing exercise.

Example 18. The method of examples 16 or 17, wherein the exercise is a first exercise and the pulmonary function of the patient comprises a first pulmonary function, the method further comprising: prompting, by the processing circuitry and via the user interface, the patient to perform a second exercise according to the periodic schedule; controlling, by the processing circuitry, the sensing circuitry to sense the one or more physiological signals of the patient of the patient while the patient performs the second exercise; based on the one or more physiological signals sensed while the patient performs the second exercise, determining, by the processing circuitry, a second pulmonary function of the patient; based on the first pulmonary function and the second pulmonary function of the patient, determining, by the processing circuitry, an overall pulmonary function of the patient; and outputting, by the processing circuitry, an indication indicative of the overall pulmonary function of the patient for user review.

Example 19. The method of example 18, wherein the first exercise comprises a deep breathing exercise, and wherein the second exercise comprises a walking exercise.

Example 20. The method of any of examples 16–19, wherein determining the pulmonary function of the patient comprises determining, by the processing circuitry, one or more indices of pulmonary function, wherein the one or more indices of pulmonary function comprise one or more of: a forced expiratory volume in one second (FEV1); a forced vital capacity (FVC); an FEV1 to FVC ratio; a forced mid-expiratory flow (FEF 25-75); or a peak expiratory flow (PEF).

Example 21. The method of any of examples 16–20, further comprising: determining, by the processing circuitry, the pulmonary function of the patient based on the one or more physiological signals sensed continuously according to a periodic schedule.

Example 22. The method of any of examples 16–21, wherein the user comprises one or more of: a clinician; a caregiver; or the patient.

Example 23. The method of example 22, further comprising: comparing, by the processing circuitry, the pulmonary function of the patient to one or more corresponding criteria; and based on the comparison, determining, by the processing circuitry, whether to output the indication indicative of the pulmonary function of the patient to the patient, the caregiver, or the clinician.

Example 24. The method of any of examples 16–23, wherein the output indicative of the pulmonary function of the patient comprises one or more of: a trend in pulmonary function; a suggestion to adjust a patient treatment; or an indication to use a rescue inhaler.

Example 25. The method of any of examples 16–24, further comprising: adjusting, by the processing circuitry, the periodic schedule based on one or more of: the pulmonary function of the patient; an overall health of the patient; or one or more environmental factors.

Example 26. The method of any of examples 16–25, further comprising determining, by the processing circuitry, a quality level associated with one or more of the one or more physiological signals or the one or more parameters; and based on the quality level of the one or more physiological signals meeting one or more criteria, determining, by the processing circuitry, the pulmonary function of the patient.

Example 27. The method of any of examples 16–26, wherein the one or more physiological signals include one or more of: an impedance measurement; an electromyography (EMG) signal; an electrocardiogram (ECG) signal; a heart rate signal; a photoplethysmography (PPG) signal; a tissue oxygen saturation signal; a peripheral oxygen saturation signal; an accelerometer signal; or a pressure signal.

Example 28. The method of any of examples 16–27, wherein outputting the indication indicative of the pulmonary function of the patient comprises outputting an indication of a chronic obstructive pulmonary disease (COPD) status of the patient.

Example 29. The method of any of examples 16–28, wherein the user interface comprises a user interface of a user device.

Example 30. The method of any one or more of examples 16–29, wherein the IMD comprises a housing configured for subcutaneous implantation, the housing having a length, a width, and a depth, wherein the length is greater than the width, and the width is greater than the depth, and the length is within a range from 40 millimeters (mm) to 60 mm, and wherein the IMD comprises a plurality of sensors on the housing configured to sense the one or more physiological signals.

Example 31. A non-transitory computer-readable medium storing instructions that when executed cause processing circuitry to: prompting, via a user interface, a patient to perform an exercise according to a periodic schedule; control sensing circuitry of an implantable medical device (IMD) of a medical device system comprising the processing circuitry to sense one or more physiological signals of the patient while the patient performs the exercise; based on the one or more physiological signals sensed while the patient performs the exercise, determine a pulmonary function of the patient; and output an indication indicative of the pulmonary function of the patient for user review.

Various examples have been described. These and other examples are within the scope of the following claims.

What is claimed is:

1. A system comprising:
an implantable medical device (IMD) comprising sensing circuitry configured to sense one or more physiological signals of a patient; and
processing circuitry configured to:
prompt, via a user interface, the patient to perform an exercise according to a periodic schedule;
control the sensing circuitry to sense the one or more physiological signals of the patient while the patient performs the exercise;
based on the one or more physiological signals sensed while the patient performs the exercise, determine a pulmonary function of the patient; and
output an indication indicative of the pulmonary function of the patient for user review.

2. The system of claim 1, wherein the exercise is a first exercise and the pulmonary function of the patient comprises a first pulmonary function, and wherein the processing circuitry is further configured to:
prompt, via the user interface, the patient to perform a second exercise according to the periodic schedule;
control the sensing circuitry to sense the one or more physiological signals of the patient of the patient while the patient performs the second exercise;
based on the one or more physiological signals sensed while the patient performs the second exercise, determine a second pulmonary function of the patient;
determine, based on the first pulmonary function and the second pulmonary function of the patient, an overall pulmonary function of the patient; and
output an indication indicative of the overall pulmonary function of the patient for user review.

3. The system of claim 2, wherein the first exercise comprises a deep breathing exercise, and wherein the second exercise comprises a walking exercise.

4. The system of claim 1, wherein to determine the pulmonary function of the patient, the processing circuitry is configured to determine one or more indices of pulmonary function, wherein the one or more indices of pulmonary function comprise one or more of:
a forced expiratory volume in one second (FEV1);
a forced vital capacity (FVC);
an FEV1 to FVC ratio;
a forced mid-expiratory flow (FEF 25-75); or
a peak expiratory flow (PEF).

5. The system of claim 1, wherein the processing circuitry is further configured to:
determine the pulmonary function of the patient based on the one or more physiological signals sensed continuously according to a periodic schedule.

6. The system of claim 1, wherein the output indicative of the pulmonary function of the patient comprises one or more of:
a trend in pulmonary function;
a suggestion to adjust a patient treatment; or
an indication to use a rescue inhaler.

7. The system of claim 1, wherein the processing circuitry is configured to:
adjust the periodic schedule based on one or more of:
the pulmonary function of the patient;
an overall health of the patient; or
one or more environmental factors.

8. The system of claim 1, wherein the processing circuitry is further configured to:
determine a quality level associated with one or more of the one or more physiological signals; and
based on the quality level of the one or more physiological signals meeting one or more criteria, determine the pulmonary function of the patient.

9. The system of claim 1, wherein the indication indicative of the pulmonary function of the patient comprises an indication of a chronic obstructive pulmonary disease (COPD) status of the patient.

10. The system of claim 1, wherein the IMD comprises a housing configured for subcutaneous implantation, the housing having a length, a width, and a depth, wherein the length is greater than the width, and the width is greater than the depth, and the length is within a range from 40 millimeters (mm) to 60 mm, and
wherein the IMD comprises a plurality of sensors on the housing configured to sense the one or more physiological signals.

11. A method comprising:
prompting, by processing circuitry of a medical device system comprising an implantable medical device (IMD) and via a user interface, a patient to perform an exercise according to a periodic schedule;
controlling, by the processing circuitry, sensing circuitry of the IMD to sense one or more physiological signals of the patient while the patient performs the exercise;
based on the one or more physiological signals sensed while the patient performs the exercise, determining, by the processing circuitry, a pulmonary function of the patient; and
outputting, by the processing circuitry, an indication indicative of the pulmonary function of the patient for user review.

12. The method of claim 11, wherein the exercise is a first exercise and the pulmonary function of the patient comprises a first pulmonary function, the method further comprising:
prompting, by the processing circuitry and via the user interface, the patient to perform a second exercise according to the periodic schedule;
controlling, by the processing circuitry, the sensing circuitry to sense the one or more physiological signals of the patient of the patient while the patient performs the second exercise;
based on the one or more physiological signals sensed while the patient performs the second exercise, determining, by the processing circuitry, a second pulmonary function of the patient;
based on the first pulmonary function and the second pulmonary function of the patient, determining, by the processing circuitry, an overall pulmonary function of the patient; and
outputting, by the processing circuitry, an indication indicative of the overall pulmonary function of the patient for user review.

13. The method of claim 12, wherein the first exercise comprises a deep breathing exercise, and wherein the second exercise comprises a walking exercise.

14. The method of claim 11, wherein determining the pulmonary function of the patient comprises determining, by the processing circuitry, one or more indices of pulmonary function, wherein the one or more indices of pulmonary function comprise one or more of:
a forced expiratory volume in one second (FEV1);
a forced vital capacity (FVC);
an FEV1 to FVC ratio;
a forced mid-expiratory flow (FEF 25-75); or
a peak expiratory flow (PEF).

15. The method of claim 11, further comprising:
determining, by the processing circuitry, the pulmonary function of the patient based on the one or more physiological signals sensed continuously according to a periodic schedule.

16. The method of claim 11, wherein the output indicative of the pulmonary function of the patient comprises one or more of:
a trend in pulmonary function;
a suggestion to adjust a patient treatment; or
an indication to use a rescue inhaler.

17. The method of claim 11, further comprising:
adjusting, by the processing circuitry, the periodic schedule based on one or more of:
the pulmonary function of the patient;
an overall health of the patient; or
one or more environmental factors.

18. The method of claim 11, further comprising
determining, by the processing circuitry, a quality level associated with one or more of the one or more physiological signals or the one or more parameters; and
based on the quality level of the one or more physiological signals meeting one or more criteria, determining, by the processing circuitry, the pulmonary function of the patient.

19. The method of claim 11, wherein outputting the indication indicative of the pulmonary function of the patient comprises outputting an indication of a chronic obstructive pulmonary disease (COPD) status of the patient.

20. A non-transitory computer-readable medium storing instructions that when executed cause processing circuitry to:
prompting, via a user interface, a patient to perform an exercise according to a periodic schedule;
control sensing circuitry of an implantable medical device (IMD) of a medical device system comprising the processing circuitry to sense one or more physiological signals of the patient while the patient performs the exercise;
based on the one or more physiological signals sensed while the patient performs the exercise, determine a pulmonary function of the patient; and
output an indication indicative of the pulmonary function of the patient for user review.
